(19) United States
(12) Patent Application Publication
(10) Pub. No.: US 2025/0086728 A1
(43) Pub. Date: Mar. 13, 2025

(54) METHODS OF PROVIDING PROCEDURE SPECIFIC INSURANCE

(71) Applicant: Stephen A. Barber, Wilmington, DE (US)

(72) Inventor: Stephen A. Barber, Wilmington, DE (US)

(21) Appl. No.: 18/828,501

(22) Filed: Sep. 9, 2024

Related U.S. Application Data
(60) Provisional application No. 63/537,435, filed on Sep. 8, 2023.

(51) Int. Cl. G06Q 40/08; G16H 10/40

(52) U.S. Cl. CPC G06Q 40/08; G16H 10/40

(57) ABSTRACT

Disclosed are methods of providing an unplanned birth insurance policy. In some cases, the method includes providing an insurance application to a contraceptive procedure patient who has undergone or who plans to undergo a contraceptive procedure. In some cases, the method further includes collecting an insurance premium from the contraceptive procedure patient. Furthermore, in some instances the method includes making an insurance payout to the contraceptive procedure patient in the event that the contraceptive procedure fails, thereby leading to a birth of a child of the contraceptive procedure patient. Other implementations are described.

### CROSS-REFERENCE TO RELATED APPLICATIONS FIELD

This application claims priority to, and the benefit of, U.S. Provisional Application No. 63/537,435 (Attorney Docket No. 35129.2), filed Sep. 8, 2023, and entitled “Methods of Providing Unplanned Birth Insurance”, the entire disclosure of which is incorporated herein by reference.

### BACKGROUND

The present disclosure relates to the general field of insurance, and more specifically to the field of insurance against the occurrence of events. In this case, the disclosure relates to unplanned births and/or pregnancies, for which there is no known insurance field at present.

Contraception, also known as birth control, encompasses a range of methods designed to prevent pregnancy. Such methods are used by individuals and couples worldwide to prevent, plan, and space apart pregnancies according to their particular desires and circumstances. Various contraceptive techniques currently exist. For example, some contraceptive techniques utilize synthetic hormones, such as progestin or a combination of progestin and estrogen, to prevent an individual from ovulating or otherwise inhibit the individual's reproductive system. Hormonal methods may include birth control pills, patches, injections, hormonal implants, and vaginal rings. Other methods of contraception include barrier methods, which may physically block sperm from reaching an egg. This category includes condoms, diaphragms, cervical caps, and other barriers. Other methods may include intrauterine devices (IUDs), such as devices that are inserted into an individual's uterus by a healthcare professional. IUDs may utilize hormones (e.g., estrogen, progestin) or non-hormonal compounds or components to inhibit pregnancy (e.g., copper or other metals to create an inhospitable environment for sperm). Further contraceptive techniques include sterilization procedures like tubal ligation and vasectomy. In some cases contraceptive methods are intended to be temporary, while in some cases they are intended to be permanent. In many cases, contraceptive techniques require the aid of a healthcare professional to perform properly.

Notwithstanding advances in the contraception industry, no contraceptive method is 100% effective. Accordingly, in at least some cases individuals become pregnant notwithstanding implementation of one or more contraceptive techniques. In many cases, such individuals rely on the contraceptive techniques when formulating short-and long-term plans, goals, budgets, and expectations, which may be disrupted by unplanned pregnancy. Furthermore, many such individuals are not prepared to support a child financially, or they are met with other challenges resulting from such unplanned pregnancy.

For at least these reasons, there are significant limitations in the contraception industry, particularly where contraception fails. These limitations remain unaddressed and create substantial financial, emotional, and other significant burdens for many individuals worldwide. The systems and methods of the present disclosure address and overcome these limitations.

### SUMMARY

Disclosed are methods of providing unique and innovative insurance products specifically designed to provide financial peace of mind for contraception patients and providers.

In some implementations, the disclosed methods protect patients from the financial burden of unintended childbirth after undergoing a contraceptive procedure, such as a vasectomy, a tubal ligation, or any other contraceptive procedure. It is often the case that, regardless of any fault on the part of a clinic or clinician, when a failed vasectomy (or other contraceptive procedure performed on a male) results in the unplanned and unintended birth of a child, the father is liable for at least some level of financial responsibility. When a failed tubal ligation (or other contraceptive procedure performed on a female) results in a pregnancy and/or the birth of a child, the mother is often faced with large financial burdens associated with the birth and rearing of the child. In any case, unplanned pregnancies and births can result in distressing financial situations (as well as physical, emotional, psychological, and other challenges). In some implementations, the disclosed methods alleviate some, if not all, of the financial burdens associated with unplanned pregnancy and birth following failed contraceptive precautions or procedures.

Moreover, despite the low likelihood of a contraceptive medical procedure failing (e.g., approximately a 1 in 2,000 chance that a vasectomy procedure will fail and result in a childbirth), the fear of this occurring is one of the top concerns of both patients and providers (often coming in second only to patient safety). Accordingly, some implementations of the methods disclosed herein provide peace-of-mind to both patients and clinicians.

In some implementations, the disclosed methods provide other substantial benefits to clinics that perform contraceptive procedures. For instance, in some cases, the disclosed methods protect clinics by providing a buffer between the patient and a potential malpractice lawsuit in the event of an unplanned birth after a clinic has performed a contraceptive procedure. Moreover, in some cases, the disclosed methods further benefit clinics by generating additional revenue for such clinics (e.g., by providing clinics a portion of the proceeds from insurance sales), and in some cases, the disclosed methods further benefit clinics by lowering their operating costs (e.g., by providing such clinics and the professionals working therein (e.g., urologists) with an avenue to negotiate a deduction on professional liability insurance cost. In some instances, the methods may provide an added benefit to clinics in the form of a competitive advantage. For example, customers may prefer a clinic through which unplanned birth insurance is offered over a competitor that does not offer such insurance.

In accordance with the foregoing, some implementations of the disclosed methods include a method of offering and providing an unplanned birth insurance policy to patients who have undergone or who plan to undergo a contraceptive procedure. While the contraceptive procedure can be any method or procedure designed to prevent the patient from having children (e.g., any contraceptive method discussed herein or otherwise), in some cases the contraceptive procedure is a vasectomy.

Although the method can include offering unplanned birth insurance to a patient in any suitable manner and at any suitable time, in some instances the method includes offering unplanned birth insurance to prospective contraceptive procedure patients at the point of sale of the contraceptive procedure (e.g., at a clinic where the contraceptive procedure is performed, alongside an advertisement for the contraceptive procedure, etc.). Additionally or alternatively, in some cases the method includes offering unplanned birth insurance directly to vasectomy patients (or prospective vasectomy patients).

In some implementations, the method includes providing an insurance application to patients who wish to obtain an unplanned birth insurance policy. Although in some cases the insurance provider provides the application to the patient directly, in some cases the clinic provides the application to the patient. Indeed, in some instances the clinic provides the application to the patient alongside one or more “new patient” packets, agreements, waivers, forms, applications, or other papers that the clinic typically requires patients to fill out before undergoing a contraceptive procedure.

Although the method can include any suitable payment or premium structure, in some cases the method includes payment by the patient of a one-time premium at a fixed rate. In this regard, the rate can be any rate suited to cover the costs of running an insurance company (including making insurance payouts), but in some cases the rate is between about $1 and $50,000, or any subrange thereof. As an example, in some cases the rate is between about $50 and $800, and in some cases the rate is approximately $300.

While the patient may pay the cost of the insurance premium in any suitable manner, in some cases the cost of the premium is integrated into the cost of the contraceptive procedure. Accordingly, in some implementations a clinic will collect the premium at the time of the procedure (or in accordance with a payment plan for the procedure), and subsequently forward the premium to the insurance provider.

In some implementations, the method includes making insurance payouts to patients in the event of an unplanned pregnancy or birth following the contraceptive procedure. For example, in some cases the method includes making a single payout of a sum of money that equals a liability limit of the insurance policy. In some cases, the method includes making payments on a schedule (e.g., by making monthly payments over a set amount of time or until the liability limit of the insurance policy has been met).

In some cases, the method includes partnering with one or more urology clinics. For example, in some cases, the method includes receiving referrals from a urology clinic in exchange for a fee. In some cases, the fee is a fixed fee, while in other cases it is a variable fee.

In some cases, the method includes providing one or members of the clinic's staff with a limited insurance license that will allow that staff member to legally explain to patients the insurance policy (along with the associated premiums, policy limits, benefits, and other features of the policy).

In some implementations, the method includes data mining, information sharing, or investing the “float” (e.g., the difference between premiums collected and claims paid out).

While the described systems and methods are set forth herein with respect to unplanned birth insurance, contraception patients, and/or contraception providers, the described systems and methods can be modified for use with any other suitable type of insurance and/or any other suitable type of situation in which a person desires and expects a certain outcome, and there is a possibility that such outcome will not be met. Indeed, in some implementations of the described systems and methods, an insurance provider works with a medical provider, a dental provider, a physical therapy provider, a personal coach, a trainer or any other provider who works with a person to help the person obtain a desired goal (e.g., a certain eyesight following Lasik surgery, a certain weight loss after liposuction or cool sculpting, a certain shade of teeth after teeth whitening, a certain appearance or outcome following plastic surgery, and/or any other desired goal. In such circumstances, if the goal is not met, the person would have an insurance claim. Thus, in some implementations, in connection with a procedure, a person is offered an insurance plan should the procedure not provide the stipulated outcome.

### DESCRIPTION OF THE DRAWINGS

The objects and features of the present disclosure will become more fully apparent from the following description and appended claims, taken in conjunction with the accompanying drawings. Understanding that these drawings depict only typical embodiments and are, therefore, not to be considered limiting of its scope, the methods will be described and explained with additional specificity and detail through the use of the accompanying drawings in which:

FIG. 1 shows a flow chart of a method of providing an unplanned birth insurance policy, in accordance with a representative embodiment of methods disclosed herein.

FIG. 2 shows a diagram of an environment in which representative embodiments of methods disclosed herein may be performed.

FIG. 3 illustrates a representative system that provides a suitable operating environment for use with some embodiments of the described systems and methods.

FIG. 4 illustrates a repetitive networked system for use with some embodiments of the described systems and methods.

### DETAILED DESCRIPTION

A description of embodiments will now be given with reference to the Figures. It is expected that the disclosed methods may take many other forms and shapes, hence the following disclosure is intended to be illustrative and not limiting, and the scope should be determined by reference to the appended claims.

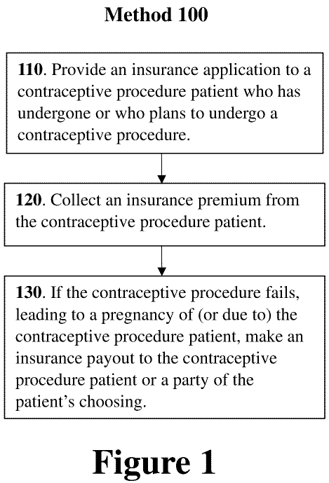

Disclosed are methods of providing unique and innovative insurance products specifically designed to provide financial peace of mind for contraception patients and providers. As shown in FIG. 1, in some embodiments, the disclosed methods include a method 100 of providing an unplanned birth insurance policy. Although the method can include any suitable element helpful in providing an unplanned birth insurance policy, in some embodiments the method includes one or more of the following: (1) (as shown in box 110) providing an insurance application to a contraceptive procedure patient who has undergone or who plans to undergo a contraceptive procedure; (2) (as shown in box 120) collecting an insurance premium from the contraceptive procedure patient; and (3) (as shown in box 130) in a case in which the contraceptive procedure fails, leading to a pregnancy and/or birth of a child of the contraceptive procedure patient, making an insurance payout to the contraceptive procedure patient.

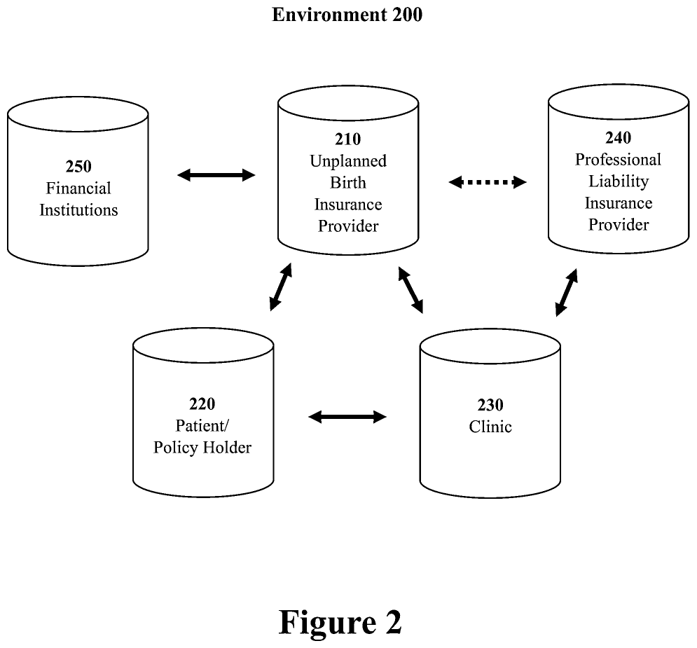

Although the method 100 may be practiced in any suitable environment, as shown in FIG. 2, in some cases the method is performed in an environment 200 involving interactions between one or more particular individuals or organizations. As an example, in some embodiments, an unplanned birth insurance provider 210 may offer and provide an unplanned birth insurance policy to one or more patients 220 who have undergone or who plan to undergo a contraceptive procedure. While the contraceptive procedure can be any method or procedure designed to prevent the patient from having children (e.g., any contraceptive method discussed herein or otherwise), in some cases the contraceptive procedure is a professional contraceptive procedure performed by a clinic 230. In this regard, the clinic may be any suitable individual or organization equipped and certified to provide medical treatment (including at least one type of contraceptive procedure), but in some embodiments the clinic is a hospital, healthcare facility, or another type of clinic.

In some embodiments, the unplanned birth insurance provider 210 interacts directly with the clinic 230. For example, in some embodiments the clinic refers the patient 220 to the unplanned birth insurance provider 210. In some embodiments, the clinic has one or more additional insurance policies from a medical malpractice insurance provider or another professional liability insurance provider 240. In some embodiments, the unplanned birth insurance provider 210 may interact (directly in indirectly) with the professional liability insurance provider 240 (e.g., in order to help clinic 230 obtain better rates). Furthermore, in some embodiments, the unplanned birth insurance provider interacts with one or more financial institutions 250 (e.g., in order to invest “float” as described in greater detail below).

The remainder of the disclosure expounds on the systems and methods encompassed herein, such as the method 100 of FIG. 1 as performed in the environment 200 of FIG. 2.

Although the method 100 can include offering unplanned birth insurance to a patient 220 in any suitable manner and at any suitable time, in some instances the method includes offering unplanned birth insurance to prospective contraceptive procedure patients at the point of sale of the contraceptive procedure (e.g., at a clinic 230 where the contraceptive procedure is performed, alongside an advertisement for the contraceptive procedure, etc.). Additionally or alternatively, in some cases the method includes offering unplanned birth insurance directly to a contraceptive procedure patient (or prospective contraceptive procedure patient). In some embodiments, the patient is a vasectomy patient (or prospective vasectomy patient).

In some implementations, the method 100 includes providing an insurance application to patients who wish to obtain an unplanned birth insurance policy (as shown in box 110). Although in some cases the insurance provider provides the application to the patient directly, in some cases the clinic provides the application to the patient. Indeed, in some instances the clinic provides the application to the patient alongside one or more “new patient” packets, agreements, waivers, forms, applications, or other papers that the clinic typically requires patients to fill out before undergoing a contraceptive procedure. Accordingly, in some embodiments, the method includes providing one or more documents to a clinic to be added to the clinic's existing new patient packet. In some embodiments, such document explains one or more insurance options (e.g., in accordance with the methods disclosed here) and provides instructions to patients for enrolling in one or more insurance plans. In some embodiments, the method further includes providing to a clinic a portal (e.g., a software portal in an electronic environment, such as over the internet or another network) to upload one or more applications or other documents. In further embodiments, the portal provides a payment system through which the patient (or the clinic, or anyone else on behalf of the patient or clinic) to send a premium or payment.

Although the method can include any suitable payment or premium structure, in some cases the method includes payment by the patient 220 of a one-time premium at a fixed rate (see box 120). In this regard, the rate can be any rate suited to cover the costs of running an insurance company (including making insurance payouts), but in some cases the rate is between about $1 and $50,000, or any subrange thereof. As an example, in some cases the rate is between about $50 and $800, and in some cases the rate is approximately $300. In some embodiments, the premium is a variable premium. Where the premium is a variable premium, the premium may be calculated based on any suitable metric, but in some embodiments the premium is calculated as a percentage of the liability limit of the insurance policy. In some embodiments, the premium is calculated depending on a number of factors of the patient, such as age of the patient, number of biological children and/or previous pregnancies of the patient, likelihood of failure of the contraceptive procedure, marital status, or any other factor that might influence the risk that failure of the contraceptive procedure will give rise to an unplanned pregnancy. In some embodiments, the premium may be paid (e.g., by the clinic, the patient, or another party) in a single payment, whereas in other embodiments the premium may be paid in accordance with a payment schedule. In some embodiments, the payment schedule aligns with an expected term of recovery from a contraceptive procedure.

While the patient 220 (or anyone else) may pay the cost of the insurance premium in any suitable manner, in some cases the cost of the premium is integrated into the cost of the contraceptive procedure. Accordingly, in some implementations the clinic 230 will collect the premium at the time of the procedure (or in accordance with a payment plan for the procedure), and subsequently forward the premium to the insurance provider 210. In some embodiments, the clinic charges and retains an additional fee for collection of the premium or referral of the patient to the insurance provider.

In some implementations, the method 100 includes making insurance payouts to patients in the event of an unplanned pregnancy or birth following the contraceptive procedure (as shown in box 130). For example, in some cases the method includes making a single payout of a sum of money that equals a liability limit of the insurance policy. In some cases, the method includes making payments on a schedule (e.g., by making monthly payments over a set amount of time or until the liability limit of the insurance policy has been met). In some cases, the payment schedule is determined by and/or adjusted by milestones in the development of the child of the patient. In some embodiments, payments are terminated in the event that the patient no longer has a financial obligation to support the child (e.g., if the child is adopted, dies, and/or is taken from the custody of the patient). In some embodiments, an extra payment is provided to the policy holder at the time of birth of the policy holder's unplanned child.

In some embodiments, insurance payouts are made for the purpose of covering the expenses of raising a child. That said, in some embodiments, insurance payouts are made for any other purpose relating to unplanned pregnancy or birth. Indeed, in some embodiments, insurance payouts are made to cover the cost of doctor's appointments, pregnancy expenses, expenses arising from emotional trauma, and/or any other costs related to pregnancy, birth, child rearing, and/or any related or semi-related expenses.

While the policy limit of the insurance policy may include any suitable amount of money for providing financial assistance to a parent, in some embodiments, the policy limit is between $5,000 and $500,000,000, or any subrange therein. For example, in some cases the policy limit is between $100,000 and $500,000. In some embodiments, the policy limit is approximately $300,000. As an illustrative example, in a case in which the policy limit is approximate $300,000, the method may include providing monthly payments of approximately $1,388.88 over 216 months, or until the $300,000 limit of liability has been met.

Although the insurance payment(s) may be provided to the patient 220 in any suitable manner, in some embodiments the unplanned birth insurance provider 210 provides one or more payments to the patient through ACH deposits, paper check processes, cash payments, credit payments, reimbursements, or payment of bills for payout(s) on approved claims.

In some embodiments, payout of claims (as shown in box 130) is conditioned on certain requirements or conditions. While the payout may depend on any condition suitable for determining whether the insurance provider should be liable for the claim, in some embodiments the condition may be one or more of the following: a period of time passing between the contraceptive procedure and the conception of the child, a period of time passing between the contraceptive procedure and the birth of the child, a period of time passing between the purchase of the policy and the conception (and/or birth) of the child, the patient undergoing periodic physical examinations, an infertility test taken after the contraceptive procedure, and/or the patient taking a paternity (or maternity) test showing that the child is the biological offspring of the policy holder. To illustrate, a full-term pregnancy is 40 weeks. Accordingly, in some embodiments, a policy holder is not eligible for payout unless enough time has passed for a percentage of the full pregnancy term to have elapsed between the procedure and the birth of the child, where the percentage is commensurate with the level of development of the child (for example, in some embodiments, the birth must occur at least 24 weeks (or any other set duration between 10 and 60 weeks) after the contraceptive procedure, or the patient may be disqualified from receiving benefits). In some embodiments, payouts are conditioned on the patient taking a paternity test showing that the patient is the father of the child. In some embodiments, the patient must take a fertility test at some point between 1 and 40 weeks after the contraceptive procedure (e.g., approximately 12 weeks after the contraceptive procedure). In some embodiments, payouts are conditioned on proof of a negative result from such a fertility test. In some embodiments, payouts are further conditioned on periodic fertility tests. In some embodiments, the method includes providing a paternity test, maternity test, fertility test, and/or another type of test to the patient, and conditioning payouts on the patient's taking such test(s) and receiving particular results (e.g., a negative fertility test showing that the patient is not fertile).

In some embodiments, payouts are conditioned on the patient not conceiving a child (or causing someone else to conceive a child) after a set period of time following the contraceptive procedure (e.g., 3 weeks, 4 weeks, 5 weeks, or any other suitable amount of time).

In some embodiments, payments are further conditioned on the patient 220 filing a claims form with the unplanned birth insurance provider 210, and/or meeting various other compliance requirements (see the foregoing paragraph), while the insurance provider works to vet the veracity of the claim. In some embodiments, the claims form can be filed via the portal by which the insurance application may be submitted.

In some cases, the method 100 includes partnering with one or more clinics 230 (e.g., urology clinics). For example, in some cases, the method includes receiving referrals from a urology clinic in exchange for a fee. In some cases, the fee is a fixed fee, while in other cases it is a variable fee. In some cases, a similar insurance product is offered to the urology clinic, such that the urology clinic receives a payout in the event of a failed procedure.

In some cases, the method includes negotiating with a professional liability insurance provider 240 on behalf of the clinic 230. Accordingly, the method may provide a buffer between the patient 220 and the clinic 230 (e.g., a buffer against a claim made against the clinic's liability or malpractice insurance). Further, in some cases, the method includes obtaining a better rate or premium for one or more other insurance products for the clinic as a result of the clinic's partnership with the unplanned birth insurance provider 210. In some cases, the partnership is exclusive (such that the clinic agrees not to offer unplanned birth insurance through another provider).

In some cases, the method includes providing one or members of the clinic's staff with a limited insurance license that will allow that staff member to legally explain to patients the insurance policy (along with the associated premiums, policy limits, benefits, and other features of the policy). In some embodiments, the method includes providing a software and/or electronic portal to partnering clinics, such that the clinics can use the portal to send patients a link to the insurance application, payment portal, or other relevant documentation. In some embodiments, the method further includes compensating the clinic (e.g., with a fixed fee of any suitable amount, such as between $1-$10,000 per patient referral, or any subrange thereof (for example, approximately $25)). In some embodiments, the compensation to the clinic is not dependent on whether the patient obtains an unplanned birth insurance policy from the unplanned birth insurance provider 210. In some embodiments, the method includes displaying advertisements for the insurance product at a clinic 230 and/or in association with one or more advertisements for one or more contraceptive procedures.

In some embodiments, the method includes sending a message, such as a text message (or any other type of letter, multimedia message, email, or other electronic or physical communication) to the patient 220. In some cases, the message includes information pertaining to unplanned birth insurance (e.g., information about premiums, payments, payouts, eligibility, singing up, or any other information discussed herein or otherwise relating to the systems and methods discussed here). In some embodiments, the message includes a link whereby the patient can apply for unplanned birth insurance, create an account on the portal, visit the portal, pay a premium, receive a payout, or otherwise interact with the systems and methods discussed herein. In some embodiments, the method includes entering into an agreement with a clinic 230, providing the clinic with access to the portal, wherein the clinic has the ability to enter the patient's personal information. In some embodiments, the method includes sending the message (e.g., a text message or another type of message) to the patient automatically upon the clinic's entering the patient's information into the portal. In some embodiments, the message is manually or automatically sent upon completion of (or before, or during) any other portion of the method, or upon any other suitable action or stimulus following which sending such a message could be desirable.

In some embodiments, the method includes performing additional activities adjacent to the offering of an insurance product in order to produce additional revenue, such as data mining, information sharing, or investing the “float” (e.g., the difference between premiums collected and claims paid out). Indeed, in some embodiments, the float may be invested in longer term investments than those available to other service providers due to the impossibility (in some embodiments) of a claim being paid out in less than the time that may elapse between a contraceptive procedure and a birth of a child resulting from failure of that contraceptive procedure.

In some embodiments, the unplanned birth insurance provider 210 serve as a producer, a holding company, and/or any other role related to adjacent revenue sources. In some embodiments, however, the unplanned birth insurance provider may partner with one or more financial institutions (which may be any institutions relating to investing, revenue, insurance, or profitability). For example, in some embodiments the insurance provider partners with a fronting company.

In some embodiments, the unplanned birth insurance provider will retain a reserve (e.g., a cash reserve) having any suitable amount of money. In some cases, the reserve is between $10,000 and $50,000,000,000 (or any subrange thereof). For example, in some cases the reserve is between $1,000,000 and $10,000,000, and in some cases the reserve is approximately $6,000,000. In some cases, the method includes holding a certain percentage of each collected premium (the float) in reserve to ensure claims may be paid upon approval. While the float may be any suitable percentage of collected premiums, in some embodiments the float is between 5% and 75% of the collected premiums (or any subrange thereof). For example, in some cases, the float is between 10-12%. In some embodiments, the method includes investing all or a portion of the float. In some embodiments, the method includes separating a portion of the float that is not eligible for immediate payout (e.g., because enough time has not elapsed following the contraceptive procedure) from a portion of the float that is eligible for immediate payout (e.g., because a sufficient amount of time has elapsed for a viable child to have been born). Some embodiments include investing the separated portions of float in different ways, or otherwise treating the separated portions of float differently from one another (e.g., in accordance with any of the dispositions of funds in the following paragraph, or any other suitable manner of managing funds).

In some embodiments, the method includes retaining a certain percentage of float (and/or of other funds) in liquid forms, such as cash and cash equivalents. Such percentage may be anywhere between 5% and 95% (or any subrange thereof), but in some embodiments the percentage is approximately 50%. This may also include cash invested in money market funds, government bonds, and other similar low-risk, highly liquid investments. Further, in some embodiments, the method includes investing a certain percentage of float (and/or other funds) in fixed-income securities, government bonds, corporate bonds, asset-backed securities, and other types of investments. Again, this percentage may be any suitable percentage (e.g., between 5% and 95% or any subrange thereof), but in some embodiments it may be approximately 40%. Further, in some embodiments, the method includes investing a certain percentage of float (and/or other funds) in alternative investments, such as private equity and hedge funds, to enhance the unplanned birth insurance provider's potential for higher returns. Again, this percentage may be any suitable percentage (e.g., between 5% and 95% or any subrange thereof), but in some embodiments it may be approximately 10%.

While the described systems and methods are set forth herein with respect to unplanned birth insurance, contraception patients, and/or contraception providers, the described systems and methods can be modified for use with any other suitable type of insurance and/or any other suitable type of situation in which a person desires and expects a certain outcome, and there is a possibility that such outcome will not be met. Indeed, in some implementations of the described systems and methods, an insurance provider works with a medical provider, a dental provider, a physical therapy provider, a personal coach, a trainer or any other provider who works with a person to help the person obtain a desired goal (e.g., a certain eyesight following Lasik surgery, a certain weight loss after liposuction or cool sculpting, a certain shade of teeth after teeth whitening, a certain appearance or outcome following plastic surgery, and/or any other desired goal. In such circumstances, if the goal is not met, the person would have an insurance claim. Thus, in some implementations, in connection with a procedure, a person is offered an insurance plan should the procedure not provide the stipulated outcome.

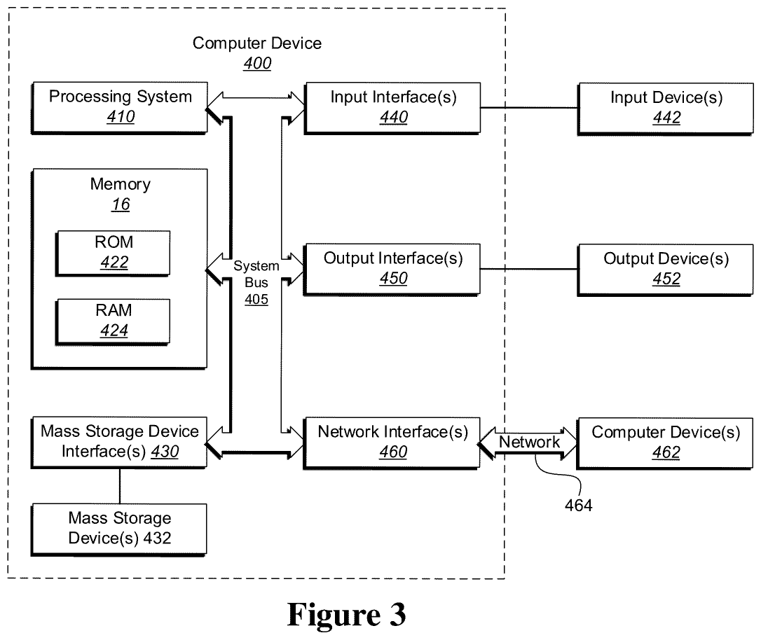

As mentioned previously, the described systems and methods can be used with, or in, any suitable operating environment and/or software. In this regard, FIG. 3 and the corresponding discussion are intended to provide a general description of a suitable operating environment in accordance with some embodiments of the described systems and methods. As will be further discussed below, some embodiments embrace the use of one or more processing (including, without limitation, micro-processing) units in a variety of customizable enterprise configurations, including in a networked configuration, which may also include any suitable cloud-based service, such as a platform as a service or software as a service.

Some embodiments of the described systems and methods embrace one or more computer readable media, wherein each medium may be configured to include or includes thereon data or computer executable instructions for manipulating data. In accordance with some embodiments, the computer executable instructions include data structures, objects, programs, routines, and/or other program modules that can be accessed by one or more processors, such as one associated with a general-purpose processing unit capable of performing various different functions or one associated with a special-purpose processing unit capable of performing a limited number of functions. In this regard, in some embodiments, the processing unit comprises a specialized processing unit that is configured for use with the described systems and methods.

Computer executable instructions cause one or more processors of the enterprise to perform a particular function or group of functions and are examples of program code means for implementing steps for methods of processing. Furthermore, a particular sequence of the executable instructions provides an example of corresponding acts that may be used to implement such steps.

Examples of computer readable media (including non-transitory computer readable media) include random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), or any other device or component that is capable of providing data or executable instructions that may be accessed by a processing unit.

With reference to FIG. 3, a representative system includes computer device 400 (e.g., one or more processors), which may be a general-purpose or special-purpose computer (or processing unit). For example, computer device 400 may be one or more processors, personal computers, notebook computers, PDAs or other hand-held devices, workstations, minicomputers, mainframes, supercomputers, multi-processor systems, network computers, processor-based consumer devices, cellular phones, tablet computers, smart phones, feature phones, smart appliances or devices, control systems, artificial intelligence, machine learning devices, neural networks, negative pressure therapy devices, mechanical closure devices, and/or the like.

In accordance with some embodiments, computer device 400 includes system bus 405, which may be configured to connect various components thereof and enables data to be exchanged between two or more components. System bus 405 may include one of a variety of bus structures including a memory bus or memory controller, a peripheral bus, or a local bus that uses any of a variety of bus architectures. Typical components connected by system bus 405 include processing system 410 and memory 420. Other components may include one or more mass storage device interfaces 430, input interfaces 440, output interfaces 450, and/or network interfaces 460, each of which will be discussed below.

In accordance with some embodiments, processing system 410 includes one or more processors, such as a central processor and optionally one or more other processors designed to perform a particular function or task. It is typically processing system 410 that executes the instructions provided on computer readable media, such as on the memory 420, a magnetic hard disk, a removable magnetic disk, a magnetic cassette, an optical disk, or from a communication connection, which may also be viewed as a computer readable medium.

Some embodiments of memory 420 include one or more computer readable media (including, without limitation, non-transitory computer readable media) that may be configured to include or includes thereon data or instructions for manipulating data, and may be accessed by processing system 410 through system bus 405. Memory 420 may include, for example, ROM 422, used to permanently store information, and/or RAM 424, used to temporarily store information. ROM 422 may include a basic input/output system (“BIOS”) having one or more routines that are used to establish communication, such as during start-up of computer device 400. RAM 424 may include one or more program modules, such as one or more operating systems, application programs, and/or program data.

One or more mass storage device interfaces 430 may be used to connect one or more mass storage devices 432 to the system bus 405. The mass storage devices 432 may be incorporated into or may be peripheral to the computer device 400 and allow the computer device 400 to retain large amounts of data. Optionally, one or more of the mass storage devices 432 may be removable from computer device 400. Examples of mass storage devices include hard disk drives, magnetic disk drives, tape drives, solid state mass storage, and optical disk drives.

Examples of solid-state mass storage include flash cards and memory sticks. A mass storage device 432 may read from and/or write to a magnetic hard disk, a removable magnetic disk, a magnetic cassette, an optical disk, or another computer readable medium. Mass storage devices 432 and their corresponding computer readable media provide nonvolatile storage of data and/or executable instructions that may include one or more program modules, such as an operating system, one or more application programs, other program modules, or program data. Such executable instructions are examples of program code means for implementing steps for the methods disclosed herein.

One or more input interfaces 440 may be employed to enable a user to enter data (e.g., initial information) and/or instructions to computer device 400 through one or more corresponding input devices 442. Examples of such input devices include a keyboard and/or alternate input devices, such as one or more switches, buttons, dials, sensors, digital cameras, pin pads, touch screens, mice, trackballs, light pens, styluses, or other pointing devices, microphones, joysticks, game pads, scanners, camcorders, and/or other input devices. Similarly, examples of input interfaces 440 that may be used to connect the input devices 442 to the system bus 405 include a serial port, a parallel port, a game port, a universal serial bus (“USB”), a firewire (IEEE 1394), a wireless receiver, a video adapter, an audio adapter, a parallel port, a wireless transmitter, or another interface.

One or more output interfaces 450 may be employed to connect one or more corresponding output devices 452 to system bus 405. Examples of output devices include a monitor or display screen, a speaker, a wireless transmitter, a printer, and the like. A particular output device 452 may be integrated with or peripheral to computer device 400. Examples of output interfaces include a video adapter, an audio adapter, a parallel port, and the like.

One or more network interfaces 460 enable computer device 400 to exchange information with one or more local or remote computer devices, illustrated as computer devices 462, via a network 464 that may include one or more hardwired and/or wireless links. Examples of the network interfaces include a network adapter for connection to a local area network (“LAN”) or a modem, BLUETOOTH™, Wi-Fi, a cellular connection, a wireless link, or another adapter for connection to a wide area network (“WAN”), such as the Internet. The network interface 460 may be incorporated with or be peripheral to computer device 400.

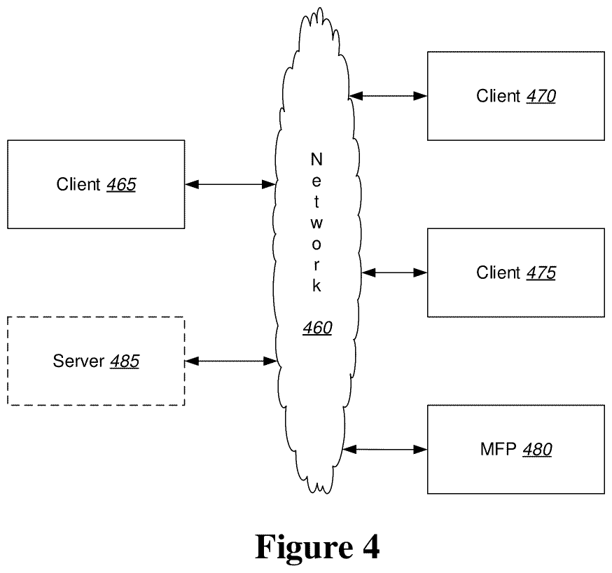

In a networked system, accessible program modules or portions thereof may be stored in a remote memory storage device. Furthermore, in a networked system computer device 400 may participate in a distributed computing environment, where functions or tasks are performed by a plurality networked computer devices. While those skilled in the art will appreciate that the described systems and methods may be practiced in networked computing environments with many types of computer system configurations, FIG. 4 represents an embodiment of a portion of a suitable system in a networked environment that includes clients (465, 470, 475, etc.) connected to a server 485 via a network 460. While FIG. 4 illustrates an embodiment that includes 3 clients connected to the network, alternative embodiments include at least one client connected to a network or many clients connected to a network. Moreover, embodiments in accordance with the described systems and methods also include a multitude of clients throughout the world connected to a network, where the network is a wide area network, such as the Internet. Accordingly, in some embodiments, the described systems and methods can allow for remote: monitoring, modification, control, training, communication, observation, adjustment, troubleshooting, data collecting, system optimization, user interaction, and/or other controlling of the described systems and methods from one or more places throughout the world.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, implementations, cases, examples, and illustrations set forth herein can be combined in absolutely any suitable manner with any other components or characteristics from one or more other embodiments, implementations, cases, examples, and illustrations described herein.

In addition, as the terms on, disposed on, attached to, connected to, coupled to, etc. are used herein, one object (e.g., a material, element, structure, member, etc.) can be on, disposed on, attached to, connected to, or otherwise coupled to another object-regardless of whether the one object is directly on, attached, connected, or coupled to the other object, or whether there are one or more intervening objects between the one object and the other object. Also, directions (e.g., front back, on top of, below, above, top, bottom, side, up, down, under, over, upper, lower, lateral, right-side, left-side, base, etc.), if provided, are relative and provided solely by way of example and for ease of illustration and discussion and not by way of limitation. Where reference is made to a list of elements (e.g., elements a, b, c), such reference is intended to include any one of the listed elements by itself, any combination of less than all of the listed elements, and/or a combination of all of the listed elements. Furthermore, as used herein, the terms a, an, and one may each be interchangeable with the terms at least one and one or more.

1. A method of providing an unplanned birth insurance policy, the method comprising:
providing an insurance application to a contraceptive procedure patient who has undergone or who plans to undergo a contraceptive procedure;
collecting an insurance premium from the contraceptive procedure patient; and
in the event that the contraceptive procedure fails, thereby leading to conception of a child of the contraceptive procedure patient, making an insurance payout to the contraceptive procedure patient.

2. The method of claim 1, further comprising offering the unplanned birth insurance policy to the contraceptive procedure patient at a clinic where the patient has or plans to undergo the contraceptive procedure.

3. The method of claim 2, further comprising providing a payment to the clinic in exchange for the offering the unplanned birth insurance policy to the contraceptive procedure patient at the clinic.

4. The method of claim 1, further comprising conditioning the making the insurance payout to the contraceptive procedure patient on at least one of the following:
the patient producing of a valid paternity test showing that the child is the contraceptive procedure patient's biological offspring;
the patient passing a test following the contraceptive procedure showing that the contraceptive procedure patient is sterile; and
a duration of time passing between the contraceptive procedure and the conception of the child, wherein the duration of time is at least 5 weeks.

5. The method of claim 1, further comprising obtaining a reduced rate on at least one of (i) professional liability insurance and (ii) medical malpractice insurance for the clinic in return for the clinic's offering the unplanned birth insurance policy to a potential patient of the clinic.
